(19) United States
(12) Patent Application Publication
(10) Pub. No.: US 2015/0051382 A1
(43) Pub. Date: Feb. 19, 2015

(54) USE OF URODILATIN FOR PREPARING A MEDICAMENT FOR THE TREATMENT OF CARDIOVASCULAR, RENAL, PULMONARY AND NEURONAL SYNDROME WHILE AVOIDING A REBOUND

(71) Applicant: CARDIOPEP PHARMA GMBH, Hannover (DE)

(72) Inventor: Wolf-Georg FORSSMANN, Kleines Wiesental (DE)

(73) Assignee: CARDIOPEP PHARMA GMBH, Hannover (DE)

(21) Appl. No.: 14/253,968

(22) Filed: Apr. 16, 2014

Related U.S. Application Data
(63) Continuation of application No. 13/635,339, filed on Nov. 15, 2012, now abandoned.

(30) Foreign Application Priority Data
Mar. 15, 2010 (EP) 10 156 516.6

(51) Int. Cl. A61K 38/22; A61K 47/26

(52) U.S. Cl. CPC A61K 38/22; A61K 47/26
USPC 530/399

(57) ABSTRACT

Use of urodilatin for preparing a medicament for the treatment of cardiovascular, renal, pulmonary and neuronal syndromes while avoiding a rebound, wherein said medicament for the delivery of urodilatin is suitable in a first quantity for a first period of at least 48 hours, followed by delivery over a second period of at least 12 hours with successive reduction of said first quantity continuously or gradually to 0 ng/kg/min.

The present invention relates to the use of urodilatin for preparing a medicament for the treatment of cardiovascular, renal, pulmonary and neuronal syndromes while avoiding a rebound.

Urodilatin is a natriuretic peptide produced in physiological quantities in the kidney (Schulz-Knappe et al., 1988), where it controls the secretion function by a closed-loop paracrine mechanism in concert with other natriuretic peptides such as ANP. BNP and CNP (Forssmann et al., 2001). However, these regulatory peptides occur systemically in the bloodstream and are general regulators of the physical functions, All in all, there are only very few organ functions that are not directly controlled or indirectly influenced by one or more of the natriuretic peptides. Thus, a deficiency due to reduced secretion or low responsiveness of the receptors (natriuretic peptide receptors) results in functional disorders and thus in essential, pathologically significant events that may end in life-threatening syndromes. Actions in concert with other systems, such as catecholamines, aldosterone, vasopressin, endothelin and many more, also play an essential role in the development of endocrine, paracrine and neuroendocrine syndromes,

Urodilatin occurs in he human blood fluid only in extremely low concentrations that are not functionally relevant. After the discovery of urodilatin, numerous papers showed that this peptide, as compared to other natriuretic peptides, surprisingly exhibits important differences that promised a better therapeutic value than that of the other natriuretic peptides (Review, Forssmann et al., 2001).

FIG. 1 shows the enzymatic degradation rate of ANP (solid circles) and urodilatin (solid diamonds) during incubation with kidney membranes, and the occurrence of proteolytic products (open circles and diamonds). The lesser degradation of urodilatin as compared to ANP is plain to see (Gagelmann et al., 1998),

Among others, these include the results of stability against endogenous proteases, such as neutral endoprotease EC-24.11, which degrades the systemically produced natriuretic peptides among others (Gagelmann et al., 1988, see also FIG. 1).

On the basis of this knowledge gained from physiological and biochemical experiments, the concept of employing urodilatin as a therapeutic substance and of testing this peptide in animal experiments (Riegger et al., 1990. Sachsenhofer et al., 1990) and also in humans (Hummel et al., 1992 and 1993. Meyer et al., 1996. Mitrovic et al., 2005 and 2006) was designed.

In the first animal experiments, an effect could actually be found that surprisingly showed a better effect in dogs as compared to other natriuretic peptides, such as ANP (Riegger et al., 1990). These effects seemed to be essentially biological activities, but not therapeutically relevant ones. Nevertheless, studies were started, hoping to alleviate renal failure after cardiac surgery or to achieve a favorable influence on chronic heart failure. The numerous minor studies did not yield remarkable success or even a breakthrough (Hummel et al., 1992 and 1993, Brenner et al., 1995. Eisner et al., 1995. Langrehr et al., 1997. Meyer et al., 1999). Although a vague therapeutic effect could be demonstrated in these experiments, a medically relevant improvement with a sustained effect was not achieved by these infusions, and a stabilization of a temporarily improved condition of the various syndromes could not be shown in a convincing and significant manner. This was in part due to the design of the studies, so that a market authorization was not achieved.

In some cases, especially after transplantation surgery involving the heart, various individual results could be collected according to which there was an improvement of renal function and of the cardiovascular parameters, which induced (Hummel et al., 1992 and 1993) a number of physicians in the field of cardiovascular surgery to initiate a request for compassionate use. Over several years, a number of observations were made in more than 2700 patients, from which some therapeutic value may be assumed.

However, neither has such value been proven by a major relevant study, nor could data sufficient for authorization be generated.

For example, in chronically stable heart failure syndromes, temporary infusions for several hours showed a temporary influence on the cardiovascular and renal parameters, which in part disappeared, however, during the infusion or immediately after the discontinuation of the treatment and underwent transition into a chronically pathological condition, for example, in NYHA III patients suffering from chronic congestive heart failure (Eisner et al., 1995). The temporary infusion with urodilatin could not influence the continuing chronic disease condition in such a way that the heart failure would regress, and the infusion for 10 hours has not been successful in terms of therapeutic progress. Although some parameters of the cardiovascular system changed in such a way that an improvement of the patient's condition could have been expected, the positive effect in part did not last even for the infusion period, and the slight to pronounced loss of action, which was interpreted as a tachyphylaxis, was already found during the infusion. In the mentioned paper by Eisner et al. (1995), this can be seen in the central venous pressure (CVP) (see FIG. 1 of the paper), which increases slightly already after 5 hours of infusion, which questions the medical value of the urodilatin infusion. The urine flow also shows a loss of action after 8 hours of infusion (see FIG. 3 of the paper), which is also considered by many experts as a tachyphylaxis.

FIG. 2 shows the effect of a 10-hour infusion with urodilatin (15 ng/kg/min-solid circles) as compared to placebo administration (open circles) on the central venous pressure (CVP) and urine flow in patients with chronic congestive heart failure. Individual comparison with preinfusion control: *p<0.05 (Eisner et al., 1995).

Therefore, the original concept of repeatedly relieving the heart by such infusions in order to improve the pathological-medical status of cardiovascular syndromes has not been pursued further. Thus, the intent to employ urodilatin in chronic heart failure has not been pursued further since a medically relevant therapeutic effect for the benefit of the patients could not be expected from these results.

However, a study dealing with bronchoconstriction in patients with moderate asthma affliction proved that a temporary biological effect from urodilatin can be shown in both healthy and diseased subjects (Flüge et al., 1999). A brief infusion shows the biologically relevant effectiveness of uradilatin on bronchoconstriction, especially as compared with other natriuretic peptides and also with additional intravenous administration of salbutamol as a state of the art medicament (albuterol, see FIG. 3).

FIG. 3 shows the time course of the forced exhalation volume per second (FEVi). Albuterol concentration: 200 ug, urodilatin infusion: 30 ng/kg/min. The combined administration (-•-) of both substances shows the strongest effect and corresponds to the maximum bronchodilation for an albuterol dose of 1250 μg (Flüge et al., 1999).

Accordingly, the therapeutic window for urodilatin is rather exactly at the intermediate dose applied of about 15 ng/kg/min. For all doses, the symptoms, morbidity and mortality were significantly improved, and most of the relevant parameters also seemed to be influenced most favorably at 15 ng/kg/min. Quite a number of essential parameters showing this tendency (Mitrovic et al., 2005 and 2006. see FIG. 4) that also demonstrated a partial persistence of the improvement in the pulmonary and renal functions in acute decompensated heart failure (ADHF) were measured (WO-A1-2006/11073).

FIG. 4 shows hemodynamic parameters for the placebo and urodilatin/ularitide in patients with chronic congestive heart failure. (A) Changes of the baseline in pulmonary capillary wedge pressure (PCWP) with significant reductions in the two highest urodilatin concentrations (15+30 ng/kg/min) as compared with the placebo group. (C) Changes of the cardiac index (CI) with significant increase of the CI for 15 and 30 ng/kg/min of urodilatin administration (Mitrovic et al., 2006).

Although a slight loss of effect was observed in part in this case too, the acute survival rate (morbidity) and the hospital dwelling time (morbidity) could be significantly and relevantly influenced for the benefit and advantage of the patients as a sign of a sustainable effect. As pointed out above, the unsatisfactory results relate to the partial loss of effect and rebound (back to worse values), for example, in the pulmonary capillary wedge pressure (PCWP) and cardiac index (CI). This can be seen in the paper by Mitrovic et al. (2006) in FIG. 2A (upper curves, PCWP) and in FIG. 2C (lower curves, CI): for both parameters, the effect is slightly reversed after 6 to 8 hours (slight increase of PCWP and partial drop of CI). Thus, the medical value is partially reduced.

One object of the present invention is to improve the application of urodilatin.

Surprisingly, it has been found that the previously practiced 24-hour infusion therapy (Mitrovic et al., 2005) can be significantly improved by the use according to the invention. The present invention for treating clinical pictures leads to significant, medically relevant improvements or healing of numerous cardiovascular, renal, pulmonary and other neuroendocrine diseases in patients.

The object of the invention is achieved by the use according to the invention of urodilatin for preparing a medicament for the treatment of cardiovascular, renal, pulmonary and neuronal syndromes while avoiding a rebound, wherein said medicament for the delivery of urodilatin is suitable in a first quantity for a first period of at least 48 hours, followed by delivery over a second period of at least 12 hours with successive reduction of said first quantity continuously or gradually to 0 ng/kg/min.

### BRIEF DESCRIPTION OF THE DRAWINGS

FIG. 1 graphically compares the enzymatic degradation rate of ANP and urodilatin.

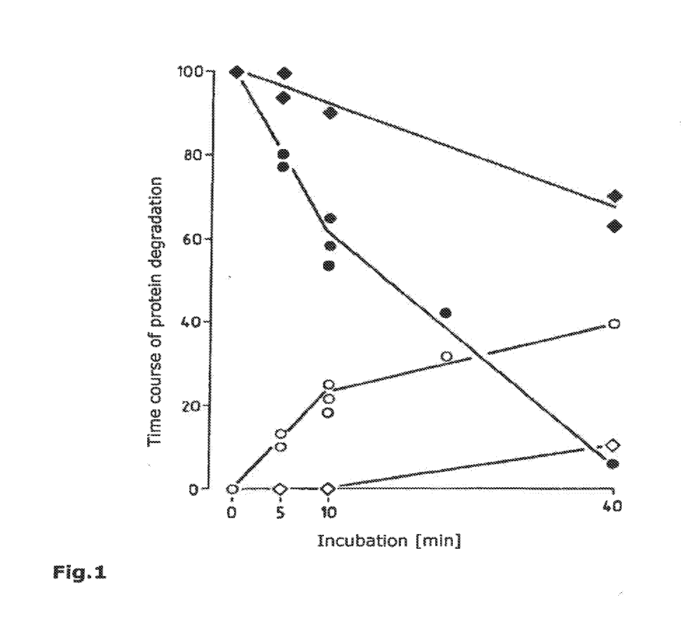

FIG. 2 graphically represents the effect of urodilatin administration as compared to placebo administration over time.

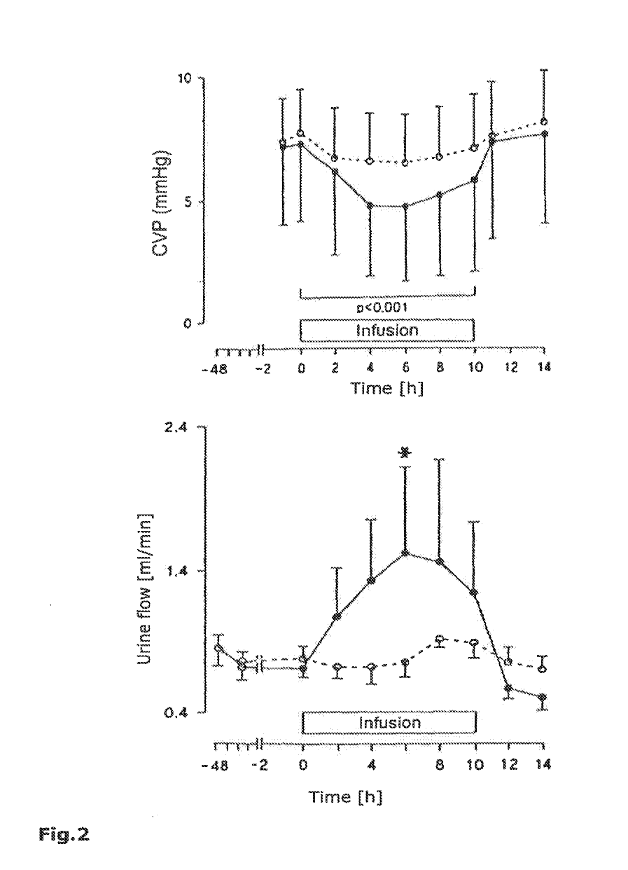

FIG. 3 graphically compares the results among urodilatin infusion, Albuterol infusion, and combined urodilatin and Albuterol infusion.

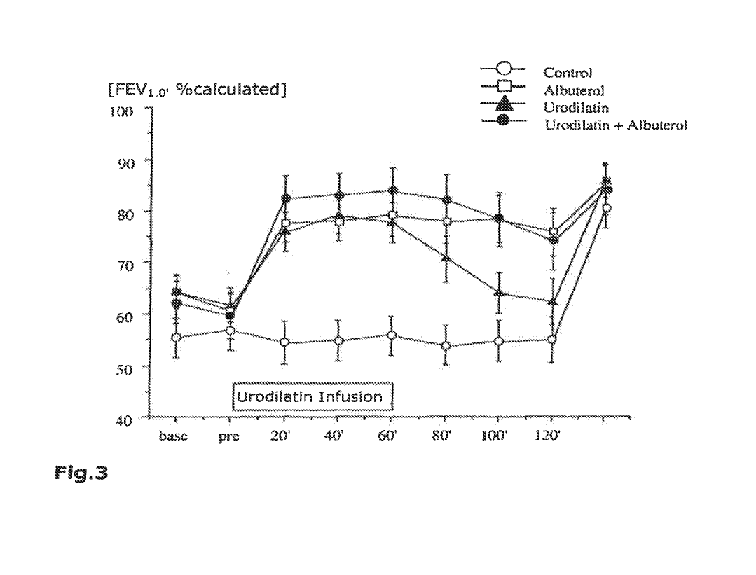

FIG. 4 graphically compares the hemodynamic parameters for urodilatin/ularitide and a placebo in patients with chronic congestive heart failure over time.

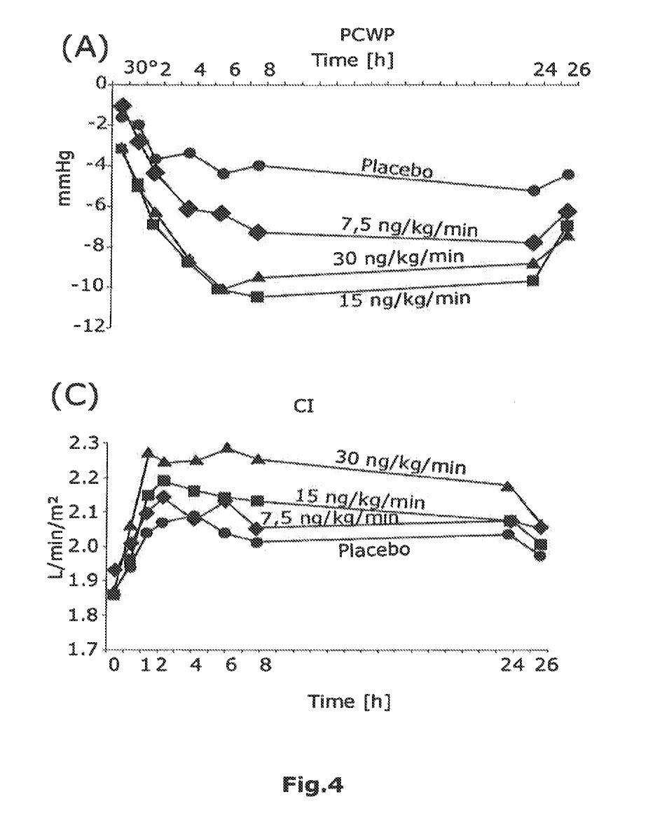

FIG. 5 graphically compares the effects of urodilatin administration at different dosages over time.

In one embodiment of the invention, said first period is from 48 hours to 120 hours, from 48 hours to 96 hours, from 48 hours to 72 hours, from 48 hours to 60 hours, from 72 hours to 96 hours, from 72 hours to 120 hours, or from 96 hours to 120 hours.

In another embodiment of the invention, said second period is from 12 hours to 72 hours, from 12 hours to 48 hours, from 12 hours to 36 hours, from 12 hours to 24 hours. from 24 hours to 72 hours, from 24 hours to 48 hours, from 24 hours to 36 hours, from 36 hours to 48 hours, from 36 hours to 72 hours, or from 48 hours to 72 hours.

The successive reduction of the first quantity of urodilatin is advantageously effected from 15 ng/kg/min to 12.5 ng/kg/min after 4 hours, to 10.0 ng/kg/min after 8 hours, to 7.5 ng/kg/min after 12 hours, to 5.0 ng/kg/min after 16 hours, to 2.5 ng/kg/min after 20 hours, and to 0 ng/kg/min after 24 hours.

In another embodiment, said first quantity is a 7.5 ng/kg/min, a 10 ng/kg/min or ≦20 ng/kg/min, especially 15 ng/kg/min.

The medicament may contain mannitol. In particular, the concentration of mannitol is about ten times that of urodilatin, and/or the medicament is an aqueous solution of about 0.9% saline in which mannitol and urodilatin are dissolved.

The use of urodilatin according to the invention relates to cardiovascular, renal, pulmonary and neuronal syndromes, especially those selected from the group consisting of heart diseases, especially acute decompensated heart failure (ADHF), acute myocardial infarction as well as acute cardiac dysrhythmia; lung diseases, especially acute asthma and acute pulmonary hypertension (APH), pulmonary edema: kidney diseases, especially imminent acute renal failure (ARF), especially in major cardiac surgery, such as CABG (coronary-arterial bypass grafting), surgery of heart valves or heart transplantations; diseases of the sensory organs, especially in acute glaucoma of the eye, and vessel-related forms of the tinnitus syndrome in the inner ear.

The invention is further illustrated by means of the following Examples.

### Example 1

### Duration and Optimization of Infusion Therapy

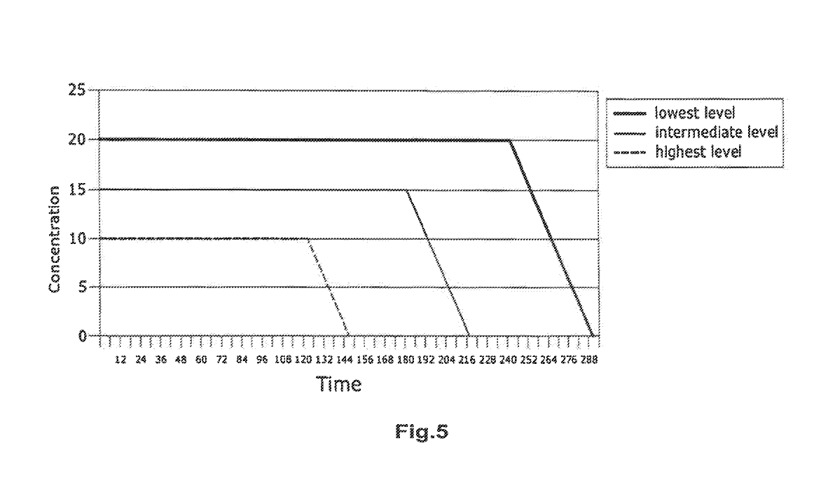

Exactly how the optimized infusion is to be performed results among others from a study performed on patients with decompensated heart failure. Infusions of 15 ng/kg of body weight/min were examined in arbitrarily assigned patients undergoing a state of the art treatment with placebo (mock infusion) or urodilatin (ularitide). Within the first 24 hours after admission to the intensive care unit, 200 patients were continuously infused 15 ng/kg of body weight/min into an arm vein. The time of the first infusion start is assigned as t₀. Ularitide is administered with a 50 ml perfusion syringe. Each vial of ularitide contains 1 mg of lyophilizate in which 10 mg of mannitol is dissolved in 5 ml of 0.9% saline, which is injected into a perfusion syringe. Subsequently, the perfusion syringe is filled with 0.9% saline to 50 ml. The flow rate is adjusted according to the corresponding formula: infusion rate ml/h)=body weight (kg)×0.03 (see Table). Placebo: The preparation of the final perfusion syringe solution and the application schedule are identical with the previous description. The dosage adapted to the body weight is adjusted according to the following criteria: All patients having a body weight of between 120 kg and 50 kg are treated with the same dosage. The minimum treatment time is at least 48 hours (followed by a 24-hour gradual withdrawal phase, see below), so that the infusion takes at least 48 hours and a maximum of 10 days. Depending on the therapeutic success, the dosage is increased to 20 ng/kg of body weight/min or reduced to 10 ng/kg of body weight/min (FIG. 5), or discontinued within 24 hours by beginning the gradual withdrawal phase:

TABLE 1
Dose reduction plan:

| | | | | 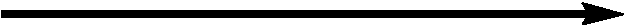 | | | | |
|---|---|---|---|---|---|---|---|---|
| Inclusion | Start on | Stop on | After stop | Reduction | | | | End of |
| period | day 1 | day 2-10 | on day 2-10 | period | | | | infusion |
| T₋₀ | T₊₀ | Tₛₜₒₚ | Tₛₜₒₚ + 4 h | Tₛₜₒₚ + 8 h | Tₛₜₒₚ + | Tₛₜₒₚ + | Tₛₜₒₚ + | T_{end} |
| | | | | | 12 h | 16 h | 20 h | |
| 0.0 ng/ | 15 ng/ | 15 ng/ | 12.5 ng/ | 10.0 ng/ | 7.5 ng/ | 5.0 ng/ | 2.5 ng/ | 0.0 ng/ |
| kg/min | kg/min | kg/min | kg/min | kg/min | kg/min | kg/min | kg/min | kg/min |

Example of a concept of a novel therapy strategy in urodilatin administration. Instead of a complete infusion stop, the total dose is successively reduced over one day after the therapy time of 2-10 days.

FIG. 5 shows a variable dose range (10, 15 and 20 ng/kg/min) for the concept of the therapy strategy according to the invention in urodilatin administration. At any rate, the dose is not discontinued as usual, but gradually withdrawn in order to avoid rebound effects.

### REFERENCES

Brenner P, Meyer M, Reichenspurner H, Meiser B, Muller R, Mentz P, Schulz-Knappe P, Überbacher H J, Kreuzer E, Überführ P, Guder W G, Wenzlaff M, Teschemacher H, Reichert B. Forssmann W G (1995). Significance of prophylactic Urodilatin (INN: Ularitide) infusion for the prevention of acute renal failure in patients after heart transplantation, Eur J Med Res 1:137-143.

Eisner O, Muders F, Muntze A, Kromer E P, Forssmann W G, Riegger G A J (1995). Efficacy of prolonged infusion of urodilatin [ANP-(95-126)] in patients with congestive heart failure. Am Heart J 129:766-773.

Flüge T. Forssmann W G, Kunkel G. Schneider B, Mentz P, Forssmann K, Meyer M (1999). Bronchodilation using combined urodilatin-albuterol administration in asthma: a randomized, double-blind, placebo-controlled trial. Eur J Med Res 4:411-415.

Forssmann W G, Meyer M, Forssmann K (2001). The renal urodilatin system: clinical implications. Cardiovasc Res 51:450-462.

Gagelmann M, Hock D, Forssmann W G (1988) Urodilatin (CDD/ANP-95-126) is not biologically inactivated by a peptidase from dog kidney cortex membranes in contrast to atrial natriuretic peptide/cardiodilatin (alpha-hANP/CDD-99-126). FEBS Letters 233:249-254.

Hummel M, Kuhn M, Bub A, Mann B, Schneider B, von Eickstedt K W, Hetzer R, Forssmann W G (1992). Urodilatin: a new peptide with beneficial effects in the postoperative therapy of cardiac transplant recipients. Clin Investig 70:674-682.

Hummel M, Kuhn M, Bub A, Mann B, Schneider B, von Eickstedt K W, Forssmann W G, Hetzer R (1993). Urodilatin, a new therapy to prevent kidney failure after heart transplantation. J Heart Lung Transplant 12:209-218.

Langrehr J M, Kahl A, Meyer M, Neumann U, Knoop M, Jonas S, Steinmuller T, Bechstein W O, Frei U, Forssmann W G, Neuhaus P (1997). Prophylactic use of low-dose urodilatin for prevention of renal impairment following liver transplantation: a randomized placebo-controlled study. Clin Transplant 11:593-598.

Meyer M, Richter R, Brunkhorst R. Wrenger E, Schulz-Knappe P, Kist A, Mentz P, Brabant E G, Koch K M, Rechkemmer G, Forssmann W G (1996). Urodilatin is involved in sodium homeostasis and exerts sodium-state-dependent natriuretic and diuretic effects. Am J Physiol 271: F489-F497.

Meyer M, Pfarr E, Schirmer G Überbacher H J, Schöpe K, Böhm E, Flüge T, Mentz P. Scigalla P. Forssmann W G (1999). Therapeutic use of the natriuretic peptide ularitide in acute renal failure. Ren Fail 21:85-100

Mitrovic V, Lüss H, Nitsche K, Forssmann K. Maronde E, Fricke K, Forssmann W G, Meyer M (2005). Effects of the renal natriuretic peptide urodilatin (ularitide) in patients with decompensated chronic heart failure: a double-blind, placebo-controlled, ascending-dose trial. Am Heart J 150:1239.e1-1239.e8.

Mitrovic V, Seferovic P M, Simeunovic D. Ristic A D, Miric M, Moiseyev V S, Kobalava Z, Nitsche K. Forssmann W G, Lüss H, Meyer M (2006). Haemodynamic and clinical effects of ularitide in decompensated heart failure. Eur Heart J 27:2823-2832.

Riegger J A G, Eisner D, Forssmann W G, (1990). Effects of ANP-(95-126) in dogs before and after induction of heart failure. Am J Physiol 259:H1643-H1648.

Sachsenhofer, Rase A, Weidmann P (1990). Urodilatin, a natriuretic factor from kidney, can modify renal and cardiovascular function in men. Am J Physiol 259:F832-F838.

Schulz-Knappe P, Forssmann K, Herbst F, Piepkorn, Forssmann W G (1988). Isolation and structural analysis of “urodilatin”, a new peptide of the cardiodilatin-(ANP)-family, extracted from human urine. Klin Wochenschr 66:752-759.

1. Use of urodilatin for preparing a medicament for the treatment of cardiovascular, renal, pulmonary and neuronal syndromes while avoiding a rebound, wherein said medicament for the delivery of urodilatin is suitable in a first quantity for a first period of 48 hours or more, followed by delivery over a second period of 12 hours or more, in particular 24 hours or more with successive reduction of said first quantity continuously or gradually to 0 ng/kg/min.

2. The use according to claim 1, wherein said first period is from 48 hours to 120 hours, from 48 hours to 96 hours, from 48 hours to 72 hours, from 48 hours to 60 hours, from 72 hours to 96 hours, from hours to 120 hours, or from 96 hours to 120 hours.

3. The use according to claim 1, wherein said second period is from 12 hours to 72 hours, from 12 hours to 48 hours, from 12 hours to 36 hours, from 12 hours to 24 hours, from 24 hours to 72 hours, from 24 hours to 48 hours, from 24 hours to 36 hours, from 36 hours to 48 hours, from 36 hours to 72 hours, or from 48 hours to 72 hours.

4. The use according to claim 3, wherein said successive reduction of the first quantity of urodilatin is effected from 15 ng/kg/min to 12.5 ng/kg/min after 4 hours, to 10.0 ng/kg/min after 8 hours, to 7.5 ng/kg/min after 12 hours, to 5.0 ng/kg/min after 16 hours, to 2.5 ng/kg/min after 20 hours, and to 0 ng/kg/min after 24 hours.

5. The use according to claim 1, wherein said first quantity is >7.5 ng/kg/min, >10 ng/kg/min or <20 ng/kg/min, especially 15 ng/kg/min,

6. The use according to claim wherein said medicament contains mannitol.

7. The use according to claim 1, wherein the concentration of mannitol is about ten times that of urodilatin, and/or the medicament is an aqueous solution of about 0.9% saline in which mannitol and urodilatin are dissolved.

8. The use according to claim 1, wherein cardiovascular, renal, pulmonary and neuronal syndromes are selected from the group consisting of heart diseases, especially acute decompensated heart failure (ADHF), acute myocardial infarction as well as acute cardiac dysrhythmia; lung diseases, especially acute asthma and acute pulmonary hypertension (APH), pulmonary edema; kidney diseases, especially imminent acute renal failure (ARF), especially in major cardiac surgery, such as CABG (coronary-arterial bypass grafting), surgery of heart valves or heart transplantations; diseases of the sensory organs, especially in acute glaucoma of the eye, and vessel-related forms of the tinnitus syndrome in the inner ear.
